(19) United States
(12) Patent Application Publication
(10) Pub. No.: US 2026/0321425 A1
(43) Pub. Date: Oct. 15, 2026

(54) MACHINE LEARNING FOR DETECTING HYPERTENSIVE DISORDERS IN PREGNANCIES

(71) Applicant: Delfina Care Inc., San Jose, CA (US)

(72) Inventors: Isabel FULCHER, Sunnyvale, CA (US); Noam FINKELSTEIN, Sharon, VT (US); Ali EBRAHIM, San Jose, CA (US); Senan EBRAHIM, Rochester, MN (US)

(73) Assignee: Delfina Care Inc., San Jose, CA (US)

(21) Appl. No.: 18/874,700

(22) PCT Filed: Oct. 8, 2024
(86) PCT No.: PCT/US2024/050323

§ 371 Date: Dec. 13, 2024

Related U.S. Application Data
(60) Provisional application No. 63/608,028, filed on Dec. 8, 2023.

(51) Int. Cl. G16H 50/30; G06N 20/00

(52) U.S. Cl. CPC G16H 50/30; G06N 20/00

(57) ABSTRACT

Embodiments include computing systems and methods implementing machine-learning architecture on a computer comprising hardware and software components to address the shortcomings described herein. A computer implements a machine-learning architecture for predicting or predicting instances of HDPs, which includes generating an HDP risk score indicating a likelihood or probability that a patient developed HDP during the patient's pregnancy. In some implementations, the machine-learning architecture identify and suggest intervention options for reducing the HDP risk score. Moreover, the machine-learning architecture may update the HDP risk score and other outputs dynamically, as the system obtains updated or additional patient data, such as additional measurements (e.g., blood pressure) throughout the patient's pregnancy.

### CROSS-REFERENCE TO RELATED APPLICATION

This application is the US national phase of PCT Application No. PCT/US 2024/050323, filed Oct. 8, 2024, which claims the benefit of priority to U.S. Provisional Application No. 63/608,028, filed Dec. 8, 2023, which are incorporated by reference in their entireties.

### STATEMENT REGARDING FEDERALLY SPONSORED RESEARCH OR DEVELOPMENT

This invention was made with government support under Project No. R43HD 114360-01 awarded by the Eunice Kennedy Shriver National Institute of Child Health and Human Development. The government has certain rights in the invention.

### TECHNICAL FIELD

This application generally relates to machine-learning architectures for healthcare, including prediction models for early identification of pregnancy disorders; and more specifically relates to machine learning models and techniques for predicting hypertensive disorders in pregnancies.

### BACKGROUND

Disorders developed during pregnancy, such as hypertension, diabetes, and peripartum depression, can increase the risk of adverse maternal and neonatal outcomes. Hypertension in pregnancy complicates 16% of pregnancies and is the leading cause of pregnancy-related deaths in the United States. Gestational diabetes complicates about 8% of pregnancies and is associated with preterm delivery, macrosomia, and fetal malformation; in addition, 50% of women with gestational diabetes develop Type 2 diabetes after delivery. Early interventions, such as monitoring or initiation of medication or therapy, can reduce the risk of adverse outcomes and diagnoses. Thus, the ability to clinically identify patients at risk for a variety of pregnancy-related disorders in the first trimester is imperative to initiate interventions and improve pregnancy-related health outcomes.

Pregnant individuals diagnosed with a hypertensive disorder of pregnancy (HDP) are four times more likely to be readmitted after giving birth, while the infants are three times more likely to require care in a neonatal intensive care unit. Importantly, the effects of HDPs are not limited to the pregnancy itself and result in higher risk of developing cardiovascular disease later in life. Early identification of pregnancies at risk for HDPs is a public health imperative for preventing complications and long-term health consequences.

Existing clinical risk factors for HDP alone are insufficient for identifying evolving risk profiles during pregnancy. The American College of Obstetricians and Gynecologists (ACOG) provides a “Clinical Risk Assessment for Preeclampsia” consisting of a checklist of risk factors that can be used to identify patients at high risk of preeclampsia for low-dose aspirin initiation early in pregnancy. The ACOG guidelines define “risk” as a simple rules-based algorithm, meaning that a person would be classified as “high risk” if, in the clinician's estimation, the patient satisfies a subset of characteristics from a short checklist, and “low risk” if, in the clinician's estimation, the patient satisfies none or only one. This approach is overly simplistic as it does not, for example, consider the relative or comparative importance of each clinical characteristic, account for multiplicative effects when multiple conditions are met, consider risk as a continuous measure with uncertainty, and include additional clinical factors that are collected as part of standard prenatal care, such as vital signs or laboratory data.

Recent studies have shown that implementing these recommendations as a checklist remains difficult, time-consuming, and often imprecise or even inaccurate. Incidences of Hypertensive Disorders of Pregnancy (HDP), for example, doubled in the United States from 2008 to 2019. Almost a quarter of maternal deaths that occurred during delivery hospitalization had a diagnosis code for HDP documented. According to these studies, these shortcomings are caused by difficult-to-detect variations in risk-factor data in the electronic health record (EHR). Further, the checklist approach is static and does not update or account for additional pertinent information, like blood pressure (BP) or lab results, as additional information becomes available over the course of an advancing pregnancy. Early identification of patients who would benefit from interventions may reduce incidence of HDP. Current ACOG risk criteria could be improved using machine-learning (ML) software. The conventional manual checklist process could be enhanced by software implementing ML operations to identify data patterns otherwise undetectable by humans.

Detection and care of HDP using computer-based ML techniques are well-suited to model HDP risk. Current ML systems that can flexibly capture the complex relationships between HDP risk factors and HDP risk offer a potential solution. Recent studies have demonstrated that blood pressure (BP) trajectories may have utility in predicting which patients may develop HDP, such as gestational hypertension and preeclampsia. Despite these advancements, none of the existing ML systems have been successfully validated or approved for external populations, which precludes widespread adoption for patients. As such, existing efforts to implement and automate HDP detection using ML techniques are insufficient.

### SUMMARY

Current technological approaches and standards associated with healthcare data present challenges to implementing machine-learning architectures for predicting HDPs. As an example, certain industry interoperability standards for generating, storing, hosting, and sharing healthcare data may present challenges in accessing certain types of data needed for a machine-learning architecture to make predictions of HDPs by detecting evolving (e.g., increasing or decreasing) risks of HDPs.

Embodiments include computing systems and methods implementing machine-learning architecture on a computer comprising hardware and software components to address the shortcomings described herein. A computer implements a machine-learning architecture for predicting instances of HDPs, which includes generating an HDP risk score indicating a likelihood or probability that a patient will develop HDP during the patient's pregnancy. In some implementations, the machine-learning architecture identify and suggest intervention options for reducing the HDP risk score. Moreover, the machine-learning architecture may update the HDP risk score and other outputs dynamically, as the system obtains updated or additional patient data, such as additional measurements (e.g., blood pressure) throughout the patient's pregnancy.

Embodiments described herein relate to a method of using machine-learning for predicting risks of hypertensive disorders of pregnancy (HDP), the method including: generating, by a computer, a training dataset including a plurality of training records containing training health data for a plurality of patients and a corresponding plurality of training labels indicating a health status, each training record includes an indication of a training patient, a timestamp, one or more training health measurements, and a training label indicating the health status of the training patient of the training record; for each training record in a first training set of one or more training patients having a first endpoint, extracting, by the computer, a set of training features from the one or more training measurements of the training record according to the first endpoint; for each training record in a second training set of one or more training patients having a second endpoint, extracting, by the computer, the set of training features from the one or more training measurements of the training record according to the second endpoint; training, by the computer, a machine-learning architecture of a risk prediction engine to generate a risk score using the set of training features and the training labels of the first training set of training records according to the first endpoint, the set of training features and the training labels of the second set of training records according to the second endpoint; obtaining, by the computer, a plurality of patient records for a patient containing patient health data and one or more health measurements; executing, by the computer, the machine-learning architecture of the risk prediction engine to generate the risk score for the patient using a set of patient features extracted from the one or more health measurements of the patient record; identifying, by the computer, the health status of the patient based upon comparing the risk score for the patient against a disorder prediction threshold; and generating, by the computer, health report data of the patient for display at a user interface of one or more client devices, the health report data indicating the one or more health measurements and the health status of the patient.

In some aspects, the techniques described herein relate to a method, further including selecting, by the computer, from the plurality of records of the training dataset the first training set of the training records having the first endpoint and the second training set of the training records having the second endpoint, the timestamp of each training record in the first training set satisfying the first endpoint, and the timestamp of each training record in the second training set satisfying the second endpoint.

In some aspects, the techniques described herein relate to a method, wherein each training record in the plurality of training records in the training dataset includes a source device indicator for a type of source device, including at least one of a clinician device or an edge device, and wherein the set of training features and the set of patient features includes the source device indicator for the type of source device.

In some aspects, the techniques described herein relate to a method, further including identifying, by the computer, in the training dataset a bias training record for the training patient, the health status of the bias training record indicating a disorder status for the training patient, and the timestamp of the bias training record fails to satisfy a status update threshold from the timestamp of a prior training record for the patient indicating an initial instance of the disorder status, wherein the computer omits each bias training record from the training dataset.

In some aspects, the techniques described herein relate to a method, wherein each training patient includes at least two training records having at least two training measurements.

In some aspects, the techniques described herein relate to a method, further including: for a next training record for the training patient, updating, by the computer, at least a portion of the set of training features for the training patient according to the one or more training measurements of the next training record; and re-training, by the computer, the machine-learning architecture of the risk prediction engine to generate the risk score using the set of training features as updated and the training label of the next training record.

In some aspects, the techniques described herein relate to a method, further including: for a next patient record for the patient, extracting, by the computer, a set of updated features for the patient using the one or more measurements of the next patient record; and generating, by the computer, an updated health report of the patient for display at the user interface of the one or more client devices.

In some aspects, the techniques described herein relate to a method, wherein the set of patient features for the patient and the set of training features for the training patient include at least one of: maximum blood pressure, minimum blood pressure, mean blood pressure, median blood pressure, blood pressure variability, blood pressure average real variability, blood pressure coefficient of variation, proportion of measures exceeding range, or a measurement slope.

In some aspects, the techniques described herein relate to a method, wherein the one or more measurements include at least one of: a systolic blood pressure, an interpregnancy interval, a prior diagnosis, a pre-pregnancy body mass index, a diastolic measurement, or a systolic measurement.

In some aspects, the techniques described herein relate to a method, further including in response to determining that the risk score for the patient satisfies the disorder prediction threshold, executing, by the computer, the machine-learning architecture of the risk prediction engine to identify one or more intervention actions corresponding to the set of one or more features extracted for the patient.

Embodiments described herein relate to a system of using machine-learning for predicting risks of hypertensive disorders of pregnancy (HDP), the system including: a computer including at least one processor and configured to: generate a training dataset including a plurality of training records containing training health data for a plurality of patients and a corresponding plurality of training labels indicating a health status, each training record includes an indication of a training patient, a timestamp, one or more training health measurements, and a training label indicating the health status of the training patient of the training record; for each training record in a first training set of one or more training patients having a first endpoint, extract a set of training features from the one or more training measurements of the training record according to the first endpoint; for each training record in a second training set of one or more training patients having a second endpoint, extract the set of training features from the one or more training measurements of the training record according to the second endpoint; train a machine-learning architecture of a risk prediction engine to generate a risk score using the set of training features and the training labels of the first training set of training records according to the first endpoint, the set of training features and the training labels of the second set of training records according to the second endpoint; obtain a plurality of patient records for a patient containing patient health data, one or more health measurements, and a training label indicating the health status of the training patient for the training record; execute the machine-learning architecture of the risk prediction engine to generate the risk score for the patient using a set of patient features extracted from the one or more health measurements of the training record; identify the health status of the patient based upon comparing the risk score for the patient against a disorder prediction threshold; and generate health report data of the patient for display at a user interface of one or more client devices, the health report data indicating the one or more health measurements and the health status of the patient.

In some aspects, the techniques described herein relate to a system, wherein the computer is further configured to: select, from the plurality of records of the training dataset, the first training set of the training records having the first endpoint and the second training set of the training records having the second endpoint, the timestamp of each training record in the first training set satisfying the first endpoint and the timestamp of each training record in the second training set satisfying the second endpoint.

In some aspects, the techniques described herein relate to a system, wherein each training record in the plurality of training records in the training dataset includes a source device indicator for a type of source device, including at least one of a clinician device or an edge device, and wherein the set of training features and the set of patient features includes the source device indicator for the type of source device.

In some aspects, the techniques described herein relate to a system, wherein the computer is further configured to: identify in the training dataset a bias training record for the training patient, the health status of the bias training record indicating a disorder status for the training patient, and the timestamp of the bias training record fails to satisfy a status update threshold from the timestamp of a prior training record for the patient indicating an initial instance of the disorder status, wherein the computer omits each bias training record from the training dataset.

In some aspects, the techniques described herein relate to a system, wherein each training patient includes at least two training records having at least two training measurements.

In some aspects, the techniques described herein relate to a system, wherein the computer is further configured to: for a next training record for the training patient, update at least a portion of the set of training features for the training patient according to the one or more training measurements of the next training record; and re-train the machine-learning architecture of the risk prediction engine to generate the risk score using the set of training features as updated and the training label of the next training record.

In some aspects, the techniques described herein relate to a system, wherein the computer is further configured to: for a next patient record for the patient, extract a set of updated features for the patient using the one or more measurements of the next patient record; and generate an updated health report of the patient for display at the user interface of the one or more client devices.

In some aspects, the techniques described herein relate to a system, wherein the set of patient features for the patient and the set of training features for the training patient include at least one of: maximum blood pressure, minimum blood pressure, mean blood pressure, median blood pressure, blood pressure variability, blood pressure average real variability, blood pressure coefficient of variation, proportion of measures exceeding range, or a measurement slope.

In some aspects, the techniques described herein relate to a system, wherein the one or more measurements include at least one of: a systolic blood pressure, an interpregnancy interval, a prior diagnosis, a pre-pregnancy body mass index, a diastolic measurement, or a systolic measurement.

In some aspects, the techniques described herein relate to a system, wherein the computer is further configured to in response to determining that the risk score for the patient satisfies the disorder prediction threshold, execute the machine-learning architecture of the risk prediction engine to identify one or more intervention actions corresponding to the set of one or more features extracted for the patient.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 shows components of a system for predicting and caring for hypertensive disorders of pregnancies (HDPs), according to an embodiment.

FIG. 2 depicts dataflow amongst components of a system for predicting and caring for HDPs, according to an embodiment.

FIG. 3 depicts an example of a user interface displaying health report data, according to an embodiment.

FIG. 4 shows operations of a computer-implemented method for training a machine-learning model of a machine-learning architecture of a disorder risk prediction engine for predicting HDP or detecting evolving risks of potential instances of HDP, according to an embodiment.

FIG. 5 shows operations of a computer-implemented method for predicting HDP or detecting evolving risks of potential instances of HDP according to patient data using a trained machine-learning model of a machine-learning architecture of a disorder risk prediction engine, according to an embodiment.

### DETAILED DESCRIPTION

Reference will now be made to the illustrative embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to a person skilled in the relevant art and having possession of this disclosure, are to be considered within the scope of the invention.

As mentioned, HDPs are a leading cause of pregnancy-related death in the United States. Specific interventions, such as nutrition counseling and prophylactic aspirin use, are known to prevent the onset and exacerbation of HDP. However, current approaches to identify patients early in pregnancy are limited due to challenges with collating patient data from the EHR and low precision and recall of traditional, rules-based medical calculators or checklists. While existing software-based ML solutions can flexibly capture complex relationships between HDP risk factors, these existing solutions often only render a static prediction at a single time point and cannot update or accurately adapt predictions as additional patient-related information is collected over the course of the pregnancy.

Embodiments include computing systems and methods implementing machine-learning architecture on a computer comprising hardware and software components to address the shortcomings described herein. A computer implements a machine-learning architecture for predicting instances of HDPs or detecting evolving risk of HDP, which includes generating an HDP risk score indicating a likelihood or probability that a patient developed HDP during the patient's pregnancy. In some implementations, the machine-learning architecture identify and suggest intervention options for reducing the HDP risk score. Moreover, the machine-learning architecture may update the HDP risk score and other outputs dynamically, as the system obtains updated or additional patient data, such as additional measurements (e.g., blood pressure) throughout the patient's pregnancy.

Another problem in conventional software and ML-based approaches is the historic data or training datasets. In conventional approaches, ML models and the inputs are static, so that predictions are rendered only once, at a first prenatal care visit. Emerging research indicates that blood pressure (BP) trajectories during pregnancy are associated with the development of HDP. Embodiments described herein dynamically incorporate ongoing BP measurements after the first prenatal visit to update patients' evolving risk status, and in some case, retraining the machine-learning model. Moreover, BP measurements are typically taken at in-office prenatal care visits, which are too infrequent for dynamic model updates. Embodiments described herein incorporate at-home measures, including remote blood pressure monitoring (RBPM), which increases the frequency and validity of BP measures over the course of pregnancies. The mobile application provides a platform for RBPM to generate at-home measures using edge devices that automatically sync measurements to a patient mobile application and provider-facing electronic dashboard.

Hypertension (high blood pressure) that develops during pregnancy is associated with increased health risks to pregnant people and their infants. Lifestyle interventions, like nutrition counseling, may reduce the risk of developing hypertension for some pregnant people. Embodiments described herein use patent data capture at home from a mobile application or edge devices (sometimes referred to as Internet of Things (IOT) devices) where pregnant people monitor their blood pressure at home to help clinicians identify patients who would benefit from the interventions, potentially preventing the development of HDP and leading to healthier outcomes for expecting families. Prophylactic aspirin is one of the few interventions shown to be effective in mitigating HDP risk, but only when started early (ideally prior to 20 weeks gestation) and applied to patients who would benefit based on the patient's health and risk profile. Additionally, lifestyle interventions that have shown similar effects are resource-intensive and are most cost-effective when applied to patients who benefit the most from a risk-reduction perspective. Clinicians may be more effective at assigning hypertension workflows when guided by high-performing ML algorithms, thereby reducing the overall rate of severe HDP in their patient populations.

For ease of description and understanding, the various types of patient-related information may be referred to as patient data stored in patient records, though embodiments may store and reference the patient data in other formats or data structures.

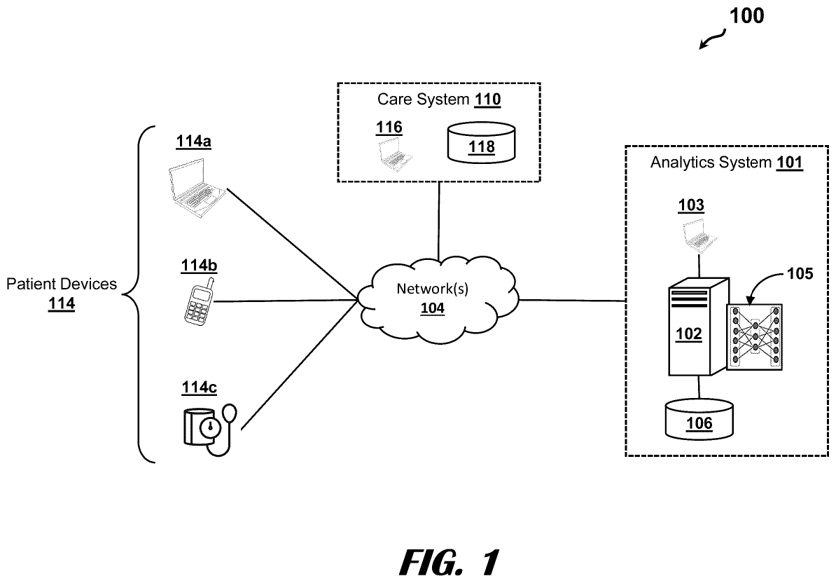

FIG. 1 shows components of a system 100 for predicting and caring for hypertensive disorders of pregnancies (HDPs), according to an embodiment. The system 100 includes an analytics system 101, care systems 110, and patient devices 114a-114c (generally referred to as patient devices 114 or a patient device 114) and provider devices 116. The components of the system 100 may communicate with one another via one or more networks 104. The analytics system 101 and care systems 110 represent computing network infrastructures 101, 110 comprising physically and logically related software and electronic devices managed or operated by various enterprise organizations, including a healthcare analytics service and healthcare providers or similar organizations (e.g., hospitals, clinics, physician office, insurance providers, research institutions). The analytics system 101 includes admin devices 103, analytics databases 106, analytics servers 102. The analytics servers 102 executes software programming for predicting the likelihood of developing HDP and caring for instances of HDP, including a machine-learning architecture of a disorder prediction engine 105. The care system 110 includes provider devices 116 and provider databases 118.

The system 100 depicted in FIG. 1 is merely an example. Embodiments may comprise additional or alternative components or omit certain components from those of FIG. 1, and still fall within the scope of this disclosure. Embodiments may include or otherwise implement any number of devices capable of performing the various features and tasks described herein.

The networks 104 host and conduct communications within the system 100. The networks 104 include various hardware components (e.g., switches, routers) and software components of one or more public or private networks, interconnecting the various components of the system 100. Non-limiting examples of such networks 104 may include: Local Area Network (LAN), Wireless Local Area Network (WLAN), Metropolitan Area Network (MAN), Wide Area Network (WAN), and the Internet. The communication over the networks 104 may be performed in accordance with various communication protocols, such as Transmission Control Protocol and Internet Protocol (TCP/IP), User Datagram Protocol (UDP), and IEEE communication protocols, implemented by the components of the networks 104, patient devices 114, and devices of the analytics system 101 and care systems 110.

The patient devices 114 may include any electronic computing devices comprising hardware and software components capable of performing the various processes and tasks described herein. Patients use the patient devices 114 to access and interact with a care application, which performs various operations that provide the patients outputs of the analytics servers 102, such as outputs of the pregnancy prediction engine 105. In the example system 100, the patient devices 114 include a patient computer 114a, a patient mobile device 114b, and a patient edge device 114c.

The patient computer 114a and the patient mobile device 114b may include any electronic device comprising a hardware (e.g., processor, non-transitory machine-readable storage medium) and software components (e.g., web browser, care application) for performing various operations for capturing measurement data, communicating with the care systems 110 or analytics system 101, and presenting an interactive user interface for the patient to review outputs of the analytics servers 102, such as patient analytics data and other care-related information. Non-limiting examples of the patient computer 114a includes a desktop computer and laptop computer, among others. Non-limiting examples of the patient mobile device 114b includes a mobile phone (or smartphone) and tablet device, among others. In some cases, the care application is natively installed on and executed by the patient device patient device 114, where the care application may contact the analytics server 102 in order to access and interact with certain features of the care application and related data. In some cases, the patient device 114 executes a web browser that accesses the features and functions of the care application, as hosted and executed by webserver software of the analytics server 102.

The patient edge device 114c includes may include an electronic device functioning as, for example, an Internet of Things (IOT) device, smart appliance, or other digital device capable of capturing measurement information. The patient edge device 114c includes any edge electronic device comprising a hardware (e.g., processor, non-transitory machine-readable storage medium) and software components for performing various operations for capturing measurement data, communicating with the patient computer 114a, patient mobile device 114b, the care systems 110 or the analytics system 101. As an example, the patient edge device 114c includes a BP cuff that connects via a wire or wirelessly with a patient mobile device 114b, captures and digitizes the BP measurements, and transmits the BP measurements to the patient mobile device 114b. The patient mobile device 114b then forwards the BP measurements to the care systems 110 or the analytics system 101, where the BP measurements are stored into the provider databases 118 or the analytics databases 106. Alternatively, the patient or care provider may manually enter the measurements into the user interface of the care application accessed or executed by the patient mobile device 114b, the patient computer 114a, or the provider device 116.

The patient device 114 transmits various types of patient data, including the measurement data and other types of patient-related information, to the care system 110 or the analytics system 101 via the one or more networks 104. The patient data may be stored into one or more storage locations, such as an analytics database 106 or provider database 118, as electronic health records (EHRs) or other form of database records.

A patient uses the patient devices 114 to access and interact with a care application and other features of the system 100. The care application may be associated with the analytics system 101 or a care system 110 that provides healthcare services to a particular patient registered with the care application. The care application includes software programming that instructs a patient device 114 to perform various operations and providing the patient outputs of the analytics servers 102, such as outputs of a disorder prediction engine 105. In some cases, the care application is natively installed on and executed by the patient device 114, where the care application may contact the analytics server 102 in order to access and interact with certain features of the care application. In some cases, the patient device 114 executes a web browser that accesses the features and functions of the care application, as hosted and executed by webserver software of the analytics server 102.

In some embodiments, the analytics system 101 hosts the care application, disorder prediction engine 105, and/or other computing services for gathering healthcare data to develop and execute software-based components of the disorder prediction engine 105. In some implementations, the care application of the analytics server 102 interacts with, and provides outputs to, the patient devices 114. Additionally or alternatively, in some implementations, the care application of the analytics server 102 interacts with, and provides outputs to, the provider devices 116 of the care systems 110 of healthcare providers.

The analytics server 102 includes any computing device comprising hardware (e.g., processors, non-transitory machine-readable storage memory) and software components (e.g., disorder prediction engine 105, webserver software) capable of performing the various processes and tasks described herein. Although FIG. 1 shows only a single analytics server 102, the analytics server 102 may include any number of computing devices. In some cases, the computing devices of the analytics server 102 may perform all or portions of the processes and benefits of the analytics server 102. The analytics server 102 may comprise computing devices operating in a distributed or cloud computing configuration and/or in a virtual machine configuration. It should also be appreciated that, in some embodiments, functions of the analytics server 102 may be partly or entirely performed by the computing devices of the care systems 110 or the patient devices 114.

The disorder prediction engine 105 includes software programming for predicting instances of HDPs occurring during a patient's pregnancy. The disorder prediction engine 105 may include software routines defining certain operational engines or functions and aspects of a machine-learning architecture (e.g., machine-learning models, machine-learning layers, among other potential operations for conditioning patient care data. The disorder prediction engine105 takes as input patient care data records, extracts features and feature vectors from the input data records, and executes programming for one or more classifier models on the extracted feature vectors to determine disorder prediction scores indicating likelihoods of pregnancy disorders. The disorder prediction engine 105 predicts a pregnancy disorder is likely when the corresponding classifier trained to predict instances of HDPs determines that the disorder prediction score satisfies a disorder prediction threshold.

The disorder prediction engine 105 operates logically in several operational phases, including a training phase and a deployment phase (sometimes referred to as “inference time”). At training time, the disorder prediction engine 105 extracts training features and training vectors from training data records to generate various predicted outputs, such as a predicted instance of HDP for a training patient. The disorder prediction engine 105 or the users may compare the predicted outputs against training labels containing expected outputs to determine a level of error. The users or machine-learning functions, such as a loss function of a loss layer, of the disorder prediction engine 105 may adjust or tune, for example, the algorithms, heuristics, data input types, hyperparameters, weights, thresholds, or other aspects of the machine-learning architecture of the disorder prediction engine 105 in order to reduce the level of error. At deployment time, the patient device 114 or the provider device 116 feeds patient data into the disorder prediction engine 105. The disorder prediction engine 105 extracts the patient's features and/or patient feature vectors from the patient data records to generate various prediction outputs.

The admin device 103 of the analytics system 101 may include any computing device comprising hardware (e.g., processors, non-transitory machine-readable storage memory) and software components capable of performing the various processes and tasks described herein. Non-limiting examples of the admin device 103 may include a personal computer (e.g., workstation computer, laptop computer), mobile device, tablet, or the like. The admin device 103 includes a user interface allowing a system administrator user to interact with the configurations of the system 100, including the configurations of the disorder prediction engine 105. The administrator may enter various configuration inputs that, for example, adjust or tune, for example, the algorithms, heuristics, data input types, weights, and thresholds, among other aspects of the functional engines of the disorder prediction engine 105.

The analytics databases 106 may be hosted on any number of computing devices comprising hardware (e.g., processors, non-transitory machine-readable storage memory) and software components capable of performing the various processes and tasks described herein. The analytics database 106 may store patient care data for patients associated with the analytics system 101 (e.g., patients treated by clinicians who utilize the disorder prediction engine 105; patients who operate a patient device 114 that executes software utilizing the disorder prediction engine 105). The analytics database 106 may include various types of electronic healthcare records (EHRs) of patients according to EHR standards, but embodiments are not so limited. In some implementations, the patient records stored in the analytics database 106 comprise additional or alternative types of data as compared to formal or standard EHR data records.

The analytics database 106 may store the various types of data from various components of the system 100. The analytics database 106 may store configurations for the disorder prediction engine 105, as received from the provider devices 116 or admin devices 103, and/or as automatically tuned by the machine-learning models and functions of the disorder prediction engine 105. In some embodiments, the analytics database 106 may further contain various instances or versions of the disorder prediction engine 105, which may be tailored by the administrators or clinicians with configurations of different care systems 110 or patients. Likewise, the provider device 116 or provider database 118 may retrieve and transmit patient data records to the analytics server 102 or the analytics database 106 via the one or more networks 104. Additionally or alternatively, the analytics server 102 or analytics database 106 may receive or retrieve the patient care data records from the analytics database 106 via the networks 104.

As mentioned, the care systems 110 include computing network infrastructures for various clinical entities, such as hospitals, physicians' offices, clinics, or research organizations, among others. The hardware and software components of the care system 110 generate and host patient care data and may access and interact with the software services (e.g., care software, disorder prediction engine 105) hosted by the analytics system 101.

The provider devices 116 include any computing device comprising hardware (e.g., processors, non-transitory machine-readable storage memory) and software components capable of performing the various processes and operations described herein. Non-limiting examples of the provider device 116 may include a personal computer (e.g., workstation computer, laptop computer), mobile device, tablet, or the like. In some cases, the provider device 116 includes a user interface allowing a care provider user (e.g., physician, healthcare worker, researcher) to interact with certain configurations of the disorder prediction engine 105 and/or the patient care data in the provider database 118 or analytics database 106. The administrator may enter various configuration inputs that, for example, adjust or tune, for example, the algorithms, heuristics, data input types, weights, and thresholds, among other aspects of the functional engines of the pregnancy prediction engine 105. The provider device 116 may, for example, enter certain configurations that indicate which types of data should be used for predicting HDP. In some cases, the provider device 116 contains patient care data (e.g., patient records, measurements, identifiers, health status information) in memory, and may provide the patient care data to the analytics system 101 as additional input patient care data inputs. The outputs generated by the analytics server 102 may be transmitted and presented to a clinician user interface of the provider device 116, so that the care provider user may review the results with the patient or other processes.

The provider databases 118 contains various types of patient data in patient data records. The provider database 118 may be hosted on any number of computing devices comprising hardware (e.g., processors, non-transitory machine-readable storage memory) and software components capable of performing the various processes and operations described herein. The provider database 118 and analytics database 106 may include various types of electronic healthcare records (EHRs) of patients according to EHR standards, but embodiments are not so limited. In some implementations, the patient records stored in the provider database 118 or the analytics database 106 comprise additional or alternative types of data as compared to formal or standard EHR data records. The provider device 116 or provider database 118 may retrieve and transmit patient data records to the analytics server 102 or the analytics database 106 via the one or more networks 104. Additionally or alternatively, the analytics server 102 or analytics database 106 may receive or retrieve the patient care data records from the provider database 118 via the networks 104.

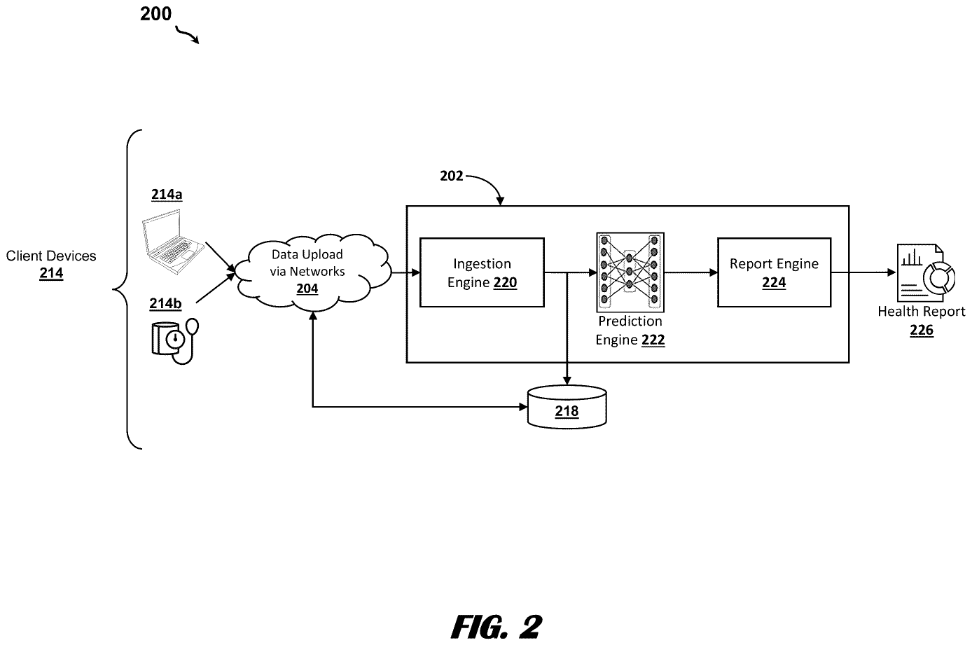

FIG. 2 depicts dataflow amongst components of a system 200 for predicting and caring for HDPs, according to an embodiment. The system 200 includes an analytics server 202, databases 218 (e.g., analytics database 106, provider database 118), one or more networks 204, and client devices 214a-214c (generally referred to as client devices 214) associated with patients or care providers, such as client computers 214a (e.g., patient computer 114a, patient mobile device 114b) and edge devices 214b (e.g., patient edge device 114c). The analytics server 202 includes software programming for performing functions or operations of an ingestion engine 220, disorder prediction engine 222, and report engine 224.

The system 200 depicted in FIG. 2 is merely an example. Embodiments may comprise additional or alternative components or omit certain components from those of FIG. 2, and still fall within the scope of this disclosure. Embodiments may include or otherwise implement any number of devices capable of performing the various features and tasks described herein.

The client computers 214a include any electronic device comprising a hardware (e.g., processor, non-transitory machine-readable storage medium) and software components (e.g., web browser, care application) for performing various operations for capturing measurement data, communicating with the analytics server 202, and presenting an interactive user interface for an end-user (e.g., care provider, patient) to review outputs of the analytics server 202, such as a health report 226. Non-limiting examples of the client computers 214a includes a desktop computer, laptop computer, tablet computer, mobile phone (or smartphone), and tablet device, among others. In some cases, the care application is natively installed on and executed by the client computers 214a, where the care application may contact the analytics server 202 in order to access and interact with certain features of the care application and related data. In some cases, the client computers 214a executes a web browser that accesses the features and functions of the care application, as hosted and executed by webserver software of the analytics server 202 or other device of the system 200.

The patient edge device 214b includes may include an electronic device functioning as, for example, an Internet of Things (IOT) device, smart appliance, or other digital device capable of capturing measurement information. The patient edge device 214b includes any edge electronic device comprising a hardware (e.g., processor, non-transitory machine-readable storage medium) and software components for performing various operations for capturing measurement data and communicating with the client computers 214a. The edge devices 214b (e.g., BP cuff, scale, body composition scanner) connects via a wire or wirelessly (e.g., Bluetooth®) with the client computer 214a, captures and digitizes the measurement data, and transmits the measurements to the client computer 214a. The client computer 214a then forwards the measurement data to the analytics server 202, where the measurement data are stored into the database 218 or other non-transitory storage media of the system 200. Alternatively, the user (e.g., patient, care provider) may manually enter the measurements into the user interface of the care application accessed or executed by the client computer 214a.

The networks 204 host and conduct communications within the system 100. The networks 204 include various hardware components (e.g., switches, routers) and software components of one or more public or private networks, interconnecting the various components of the system 200. Non-limiting examples of such networks 204 may include: Local Area Network (LAN), Wireless Local Area Network (WLAN), Metropolitan Area Network (MAN), Wide Area Network (WAN), and the Internet. The communication over the networks 204 may be performed in accordance with various communication protocols, such as Transmission Control Protocol and Internet Protocol (TCP/IP), User Datagram Protocol (UDP), and IEEE communication protocols, implemented by the components of the networks 204, client devices 214, and devices of the system 200.

The client device 214 uploads the patient data to the analytics server 202 or the databases 218 via the one or more networks 204. The client device 214 may transmit the patient data with operational instructions to the analytics server 202. For example, an administrative user may transmit the patient data and operation instructions for training or retraining aspects of the machine-learning architecture of the disorder prediction engine 222. As another example, the patient or provider may transmit the patient data with operational instructions to perform and execute the various operations of the disorder prediction engine 222 for predicting an instance HDP and the report engine 224 to generate or update the health report 226 for the patient.

The database 218 stores various types of patient related data, which may be stored in the form of patient records. For a particular patient, the databases 218 includes any number of patient records. The patient records include various data fields for various types of data. Non-limiting examples of the patient data includes various types of measurements, derived information generated by the client devices 214 or the analytics server 202, timestamps, health status indicators, health report 226 files, patient identifiers, provider identifiers, and source device identifiers indicating the client devices 214 that provided the particular patient data, among others. In some cases, the databases 218 stores the patient records with types of training data, such as training labels.

The databases 218 or other component of the system 200 store data files and programming of the machine-learning architecture of the disorder prediction engine 222. The analytics server 202 trains the machine-learning models of the machine-learning architecture, which includes updating the programming or data of the related computing files for executing the disorder prediction engine 222. Upon training (or retraining) the disorder prediction engine 222, the analytics server 202 may store the programming of these data files of the trained disorder prediction engine 222 into the databases 218 or the analytics server 202.

The analytics server 202 executes software programming for predicting HDP during a patient's pregnancy. During a training phase, the analytics server 202 executes the disorder prediction engine 222 to perform operations and functions for training a machine-learning architecture of the disorder prediction engine 222 using patient data records from the databases 218 as training records of a training dataset. During a deployment phase (sometimes referred to as “inference”), the analytics server 202 executes the disorder prediction engine 222 to perform operations and functions for generating the HDP risk score and outputting the health report 226 using the patient data records for a particular patient in the databases 218 or uploaded from the client devices 214.

The ingestion engine 220 performs various operations for ingesting and preprocessing data records for the disorder prediction engine 222. The operations include, for example, normalizing data values and generating the training dataset. For instance, in the training phase, the ingestion engine 220 (or other component of the analytics server 202) generates the training dataset by selecting or removing certain data records according to certain selection criteria for the training records.

The ingestion engine 220 may generate the training data using patient data records of patients having at least two instances of measurement data at distinct timepoints (indicated by the timestamps). The ingestion engine 220 identifies patient data records of a particular patient according to patient identifiers in the patient records of the databases 218. The ingestion engine 220 identifies selects the patient records of the patient having the patient identifier and having multiple timestamps, indicating at least two patient records and measurements for the particular patient. The ingestion engine 220 includes the selected patient records as training records of the training data records. In the way, the analytics server 202 trains the disorder prediction engine 222 on training patient records of training patients having at least two training measurements.

The ingestion engine 220 (or other component of the analytics server 202) may generate training data according to types of source devices that generated or otherwise captured the measurements of particular patient records. A patient record may include (or be associated with) a source device identifier that indicates the particular client device 214 or type of client device 214 that generated or captured the measurements of the particular patient record. For example, the patient record may include an indicator of a provider's client computer 214a in a patient data record containing measurements captured and uploaded from the provider's client computers 214a during a clinical visit. As another example, the patient record may include an indicator of a patient's edge device 214b in a patient data record containing measurements captured and generated at home by the patient using the patient's edge device 214b. In some implementations, the ingestion engine 220 selects the patient data records of patients having measurements captured by a provider's client computers 214a (clinical visit measurement) and measurements captured at the patient's edge devices 214b or client computer 214a (at-home measurement or remote measurement). In the way, the analytics server 202 trains the disorder prediction engine 222 on training patient records of training patients having clinical visit measurements and at-home measurements.

The ingestion engine 220 (or other component of the analytics server 202) may train the disorder prediction engine 222 according to time limits or time bounds applied to the measurements of the patient records. The ingestion engine 220 randomly determines certain time endpoints or time intervals for patient records. For instance, during a training phase, the ingestion engine 220 randomly selects an endpoint for sampling measurements (e.g., BP values, BP trajectory) in the training records of the training patients. In some cases, the endpoint may be selected according to a health status indicators, such as an indicator that the training patient developed an instance HDP during the pregnancy. As an example, for the training patients who went on to develop HDP having an HDP instance in the status indicator, the ingestion engine 220 may randomly select endpoints to sample measurements between 20 weeks and 1 week prior the timestamp of the HDP diagnosis; for patients who did not go on to develop HDP, the ingestion engine 220 may randomly select endpoints to sample measurements between 20 weeks and 1 week prior to delivery at a conclusion timestamp of the pregnancy. In some implementations, the ingestion engine 220 uses 20 weeks as a lower bound for the endpoint so that training patients would have at least two in-clinic BP measures.

Optionally, the ingestion engine 220 may implement time intervals using startpoints and endpoints as time-boundaries. For instance, the ingestion engine 220 assigns a 10-week startpoint and 20-week endpoint to a first set of patients and a 20-week startpoint and a 30-week endpoint to a second set of patients. In this way, the analytics server 202 trains the disorder prediction engine 222 on the first set of training patient records having measurements beginning at the 10-week startpoint and up to the 20-week endpoint and the second set of training patient records having measurements beginning at the 20-week startpoint and up to the 30-week endpoint.

The ingestion engine 220 may perform standardization operations. As shown in the example system 200, the user uploads the various types of patient data via the networks 204. The uploaded data may be in various formats or data structures. For example, a simple upload format includes Comma Separated Values (CSV) files, such as a “wide format” data table with baseline health history and pregnancy diagnoses with a row for each pregnancy, and a “long format” data table with a column for pregnancy identifier, date, timestamp, systolic value, diastolic value, and source device (e.g., in-clinic client computers 214a, at-home client computers 214a or edge devices 214b). The patient data may be uploaded or stored in the analytics server 202 or the databases 218 in various formats, such as HL7 and FHIR, among others. In some instances, the ingestion engine 220 includes mapping files for translating and converting inputted data between the various data formats or structures.

The ingestion engine 220 may perform data validation operations, to confirm and validate the quality of the input data. The ingestion engine 220 executes data-quality checks that determines whether variables of certain data types are included in the input patient data. The ingestion engine 220 may further determine whether numeric values fall within anticipated ranges preconfigured for the corresponding data fields. If the ingestion engine 220 determines that any a quality check fails, the analytics server 202 transmits a notification for display to the user indicating an error in the input patient data and related details, allowing the user to enter updates for correcting the errors in the input data.

In some embodiments, the ingestion engine 220 imposes threshold quality and amounts of data for patient records associated with a pregnancy. The ingestion engine 220 may omit or remove patient records for pregnancies in response to determining that an amount of missing data fields fails to satisfy a threshold amount of completeness, such that significant amounts of omissions are excluded from patient records and training records. For inclusion in a machine-learning architecture pipeline, the ingestion engine 220 is configured to confirm that the patient data records associated with a patient pregnancy includes a definitive health status indictor or outcome determination (e.g., HDP diagnosed or HDP not diagnosed) and at least two in-clinic BP measurements, among others (e.g., chronic hypertension status, prior diagnosis with HDP, BMI, parity).

Optionally, when uploading data, the users operating a user interface are presented an option to store the patient data in a standardized format for model fine-tuning or future analyses and/or for de-identification. Before storing, the ingestion engine 220 or other component of the system 200 performs de-identification operations, such that the ingestion engine 220 parses and stores relevant data fields and omits non-relevant data. The ingestion engine 220 may replace various identifiers with a randomly generated hash, and timestamps may be shifted based on a randomly generated number.

The disorder prediction engine 222 includes software programming for executing the machine-learning architecture for predicting HDP and detecting evolving risks of HDP according to input data records, which may include training records during training or patient records during deployment.

The disorder prediction engine 222 obtains the processed patient data records from the ingestion engine 220 and executes the machine-learning model on the input records (e.g., patient records, training records). The disorder prediction engine 222 (or other component of the analytics server 202) may extract certain features from the input record. These features may include data points in the input records, such as measurements and patient-related information.

The disorder prediction engine 222 may extract the features or a feature vector or array of values. In some cases, the training features include the data values parsed from the training records, such as the measurement values of various types of measurements (e.g., BP measurements, systolic and diastolic values). In some cases, the server references the features or values of multiple training records of the training patient to generate derived features. Non-limiting examples of derived features may include summary statistics (e.g., maximum, minimum, mean, median), BP variability (e.g., average real variability; coefficient of variation), proportion of measures exceeding range, and slope (difference between earliest and latest BP divided by gestational age), among others. The average real variability is the average of the absolute differences between consecutive BP measurements. The features may be computed separately for systolic and diastolic measures for a total of, for example, 16 derived features. The disorder prediction engine 222 considers measures relative to the endpoints. For instance, the measurements in some implementations include a systolic and/or diastolic BP measurement, an interpregnancy interval, a prior diagnosis indication, and pre-pregnancy body mass index, among others.

In some cases, the input features (e.g., training features, patient features) may include the derived features generated by the analytics server 202 using the input records, such as various the statistical values or other derived information about the patient. As the disorder prediction engine 222 obtains updated data records, the disorder prediction engine 222 may retrain the disorder prediction engine 222 as a given interval. Additionally or alternatively, the disorder prediction engine 222 may update the extracted or derived features of the patient to dynamically incorporate updated and ongoing measurements after a first prenatal visit to monitor the patient's evolving risk status.

As an example, in some cases, for each next training patient record for the training patient during the training phase, the ingestion engine 220 and disorder prediction engine 222 may ingest and extract a set of updated features for the training patient according to the one or more training measurements of the next training record. The disorder prediction engine 222 then re-trains the machine-learning architecture of the disorder prediction engine 222 for generating the risk score using the set of training features as updated and the training label of the next training record.

In some cases, for each next patient record for the patient at deployment phase, the ingestion engine 220 and disorder prediction engine 222 may ingest and extract a set of updated features for the patient using the one or more measurements of the next patient record. The disorder prediction engine 222 may then generate an updated risk score and updated health report 226 of the patient for display at the user interface of the one or more client computers 214a.

In some cases, the disorder prediction engine 222 executes the operations of the machine-learning architecture for performing model inference on the features or feature vectors extracted using the input patient data (e.g., patient records, training records), parsed or stratified by measurement type and gestational age. For measurement-type, the disorder prediction engine 222 is executed on in-clinic measures and, if available, remote measures in the input patient records. For gestational age, the disorder prediction engine 222 may compute performance metrics for a standard prenatal care visit schedule starting at predetermined time (20 weeks of gestation) and prior to term (e.g., gestational weeks 20, 24, 28, 30, 32, 34, 36) and a “combined” view that randomly samples an endpoint for each measurement (e.g., BP) trajectory to compute an overall summary metric. The combined view may be generated using patient records of timepoints between 20 weeks and 1 week prior to HDP diagnosis (for patients who develop HDP), or patent records of timepoints between 20 weeks and 1 week prior to delivery (for patients who do not develop HDP). In some cases, the disorder prediction engine 222 generates various derived features, such as various summary statistics of the various types of measures, which a report engine 224 references to generate the health report 226. In some cases, the health report 226 includes an indication of the amount of completeness or missingness of the data input record(s) of a particular patient.

The disorder prediction engine 222 uses the extracted features to create separate BP measure phenotypes for in-office measurements, and a combination of at-home and in-office measurements. To identify a number of groups (phenotypes) and group assignment for each individual patient, a classifier of the machine-learning architecture of the disorder prediction engine 222 executes an unsupervised k-means clustering or classification on the input features (e.g., patient features, training features). The disorder prediction engine 222 may standardize the patient's feature set by executing one or more transform functions, such as a Box-Cox transformation. The disorder prediction engine 222 may then execute a transform function on the feature set to produce a transform into principal components, and then extracts the components that satisfy a level of variation (e.g., 80%) for use in the classification procedure. Using the binary outcome of an HDP diagnosis (indicated by a training label or health status indicator field), the disorder prediction engine 222 tunes or fits one or more multivariate logistic regression machine-learning models for each phenotype group (e.g., in-office measures, combined measures). In some cases, the disorder prediction engine 222 may adjust or tune the logistic regression based upon, for example, race, ethnicity, and BMI. In some implementations, the disorder prediction engine 222 may perform various validation functions that compare the trained machine-learning models (e.g., using Akaike information criterion (AIC)) to identify which machine-learning model explains the greatest amount of variation.

Embodiments may implement logistic regression, though embodiments need not be so limited. Logistic regression has comparatively fewer numbers of model parameters and thus relatively easier to train and implement. The disorder prediction engine 222 may implement Bayesian inference where the disorder prediction engine 222 learns or tunes the model parameters by combining information from the training data with “prior” information. When learning the machine-learning model that incorporates at-home measurements, the disorder prediction engine 222 considers the machine-learning model parameters as strong prior information. Likewise, when learning the machine-learning model that incorporates and parameters that incorporates the derived features of the at-home measurements, the disorder prediction engine 222 considers the machine-learning model parameters as weakly informative prior distributions (e.g., represented by the Student's t-distribution enabling these parameters to be flexibly inferred).

In some implementations, training labels may indicate expected features, expected HDP risk score, or expected HDP health status. The disorder prediction engine 222 executes a loss function of a loss layer that references the training labels to determine a level of error between the predicted training features, predicted HDP risk score, or predicted HDP health status. For instance, the training labels may be used to adjust the algorithms of the disorder prediction engine 222. As an example, the loss function may compare a generated training output of the disorder prediction engine 222 against the training label indicating the ground truth to determine whether the disorder prediction engine 222 properly predicted an instance of HDP in the training patient records. The loss function may adjust the functions, heuristics, types of data input parameters, weights, or thresholds to improve the accuracy of the disorder prediction engine 222. As another example, the loss function may compare generated training outputs of the disorder prediction engine 222 (e.g., outputs of the classifier) against the training label indicating the ground truth to determine whether the disorder prediction engine 222 extracted or derived optimal features or the classifier layers of the disorder prediction engine 222 properly predicted the HDP for the training patient. The loss function may adjust the algorithm's functions, heuristics, types of data input parameters, weights, or thresholds of the disorder prediction engine 222 to improve the accuracy of the extracted or derived features extracted from the training patient's training records. In some implementations, the disorder prediction engine 222 may compute or determine which features provide the largest impact, reduce the features having a comparatively lower impact, and adjust the functions of the disorder prediction engine 222. In some cases, for example, the loss function of the disorder prediction engine 222 may implement a feature reduction approach (e.g. LASSO) to adjusting the pre disorder prediction engine 222.

The disorder prediction engine 222 may train or re-train the machine-learning model at predefined intervals or in response to receiving new training records for training patients. Likewise, at a predefined interval or in response to receiving updated patient records for the patient, the disorder prediction engine 222 may generate new or updated features, derived features, and HDP risk score for the particular patient. The dynamic parameter variables in the machine-learning model may differentiate missing values between those from a later stage in pregnancy, which have yet to be collected, and uncollected values. In some implementations, training patients in the training data records include each variables corresponding to a prior timestamp of randomly selected gestational ages marked as “yet to be collected,” enabling the machine-learning model to learn associations relevant to predictions at each stage of pregnancy.

In some embodiments, the ingestion engine 220 randomly allocates certain patient records of pregnant patients having at-home data records as potential training records. Using the training records, the loss function of the disorder prediction engine 222 may engineer maximally predictive training features from the at-home data records, taking advantage of the phenotypes, and other types of feature engineering operations. The disorder prediction engine 222 may optimize hyperparameters through cross-validation on the training records.

The disorder prediction engine 222 may determine the machine-learning model of the machine-learning architecture is trained in response to determining the level or error (or loss) satisfies a loss or training threshold. In some cases, after the model training is concluded, the disorder prediction engine 222 may evaluate or validate the trained machine-learning architecture using test patient records. In some cases, the disorder prediction engine 222 assess performance by race and ethnicity to ensure the model performs equitably and apply appropriate corrections (e.g. synthetic minority over-sampling technique).

In some embodiments, rather than using a Bayesian approach, the disorder prediction engine 222 may update or retrain the parameters of the machine-learning models of the machine-learning architecture using stochastic gradient descent, with a slow-learning rate for parameters shared with the existing model, and a high learning rate for new, at-home measurements. In some embodiments, the disorder prediction engine 222 includes a number of additive decision tree models. Such models learn a number of decision trees of restricted complexity, each of which is based on a subset of the training features. These tree models can naturally be augmented by maintaining the decision trees learned by the original model without at-home measurement data, and learning additional decision trees that include the at-home measurement features.

The outputs of the disorder prediction engine 222 may include a HDP risk score for the particular patient and an indicator of the patient's HDP health status, such as indicator that the disorder prediction engine 222 predicted an instance of HDP or did not predict an instance of HDP. The disorder prediction engine 222 generates the risk score based upon, for example, the trained parameters applied to the features extracted for the patient, or based upon a cosine similarity between the extracted feature vector of the patient and the extracted feature vector for a centroid of a cluster of training features. Classifier layers of the disorder prediction engine 222 may generate and compare the HDP risk score against a disorder prediction threshold. The disorder prediction engine 222 predicts the instance of HDP in response to the classifier determining that the HDP risk score satisfies the disorder prediction threshold.

Optionally, the disorder prediction engine 222 may be trained or programmed to identify and generated suggested interventions in response to predicting the HDP in the patient records at inference or deployment. The disorder prediction engine 222 is trained based upon correlating certain features or feature vector to one or more interventions. Non-limiting examples of potential intervention actions included in the health report 226 outputted by the disorder prediction engine 222 include aspirin initiation, additional blood and urine tests, additional remote blood pressure monitoring, antihypertensive medication, exercise programs, nutrition classes, nutrition counseling, mental health counseling, and weight counseling, among others. The outputs to the health report 226 may include the suggested or implemented interventions. In some embodiments, ongoing additional patient records may include interventions for updating the risk score or the derived features extracted for the patient.

The report engine 224 of the analytics server 202 executes software programming for generating and outputting a health report 226. The health report 226 may include machine-readable data for presentation in various formats or types. For instance, the health report 226 includes data for a user interface presented at the client devices 214 of the patient or the care provider. In some cases, the health report 226 is presented in a webpage or web-app accessible to the client computer 214a via the networks 204. In some cases, the health report 226 is presented in a user interface of a natively installed care application, installed and executed on the client computers 214a.

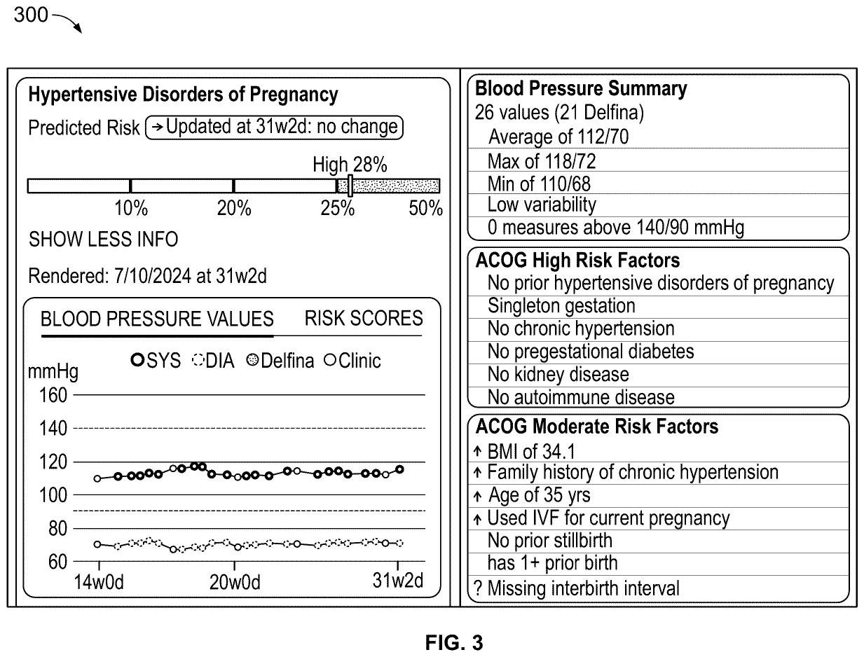

With reference to FIG. 3, which depicts an example user interface 300 presenting an example of the health report 226. The user interface 300 may be generated at a backend computing device (e.g., analytics server 202) by the disorder prediction engine 222 or other software programming associated with a care analytics platform (e.g., analytics system 101). The user interface 300 may be generated and transmitted for display at the client computers 214a. The sample health report 226 displayed in the user interface 300 includes a predicted risk score, risk score trends, and measurement data, and derived data (as derived from the measurement data).

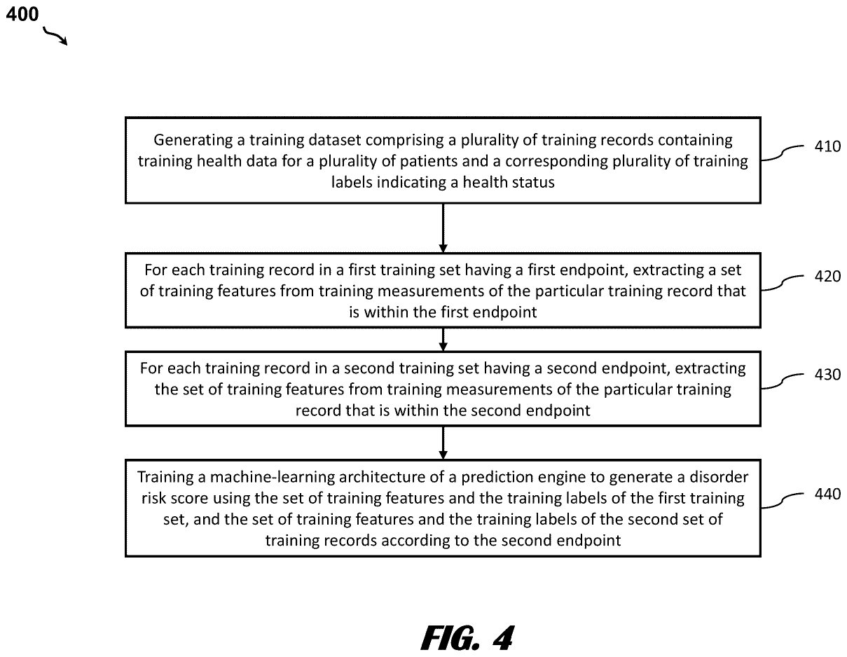

FIG. 4 shows operations of a computer-implemented method 400 for training a machine-learning model of a machine-learning architecture of a disorder risk prediction engine (e.g., disorder prediction engine 222) for predicting instances of HDP and detecting evolving risks of an HDP diagnosis, according to an embodiment. For ease of description and understanding, a server computer executes the various operations and features of the method 400, though embodiments are not so limited. Any number of computing devices may perform the operations and features of the method 400 for training the machine-learning architecture of the disorder prediction engine. Moreover, embodiments are not limited to the operations and features described in the method 400. Potential embodiments for training the machine-learning architecture of the disorder prediction engine may include any number of additional or alternative operations than those described in the method 400 and still fall within the scope of this disclosure.

At operation 410, the server generates a training dataset comprising a plurality of training records containing training health data for a plurality of patients and a corresponding plurality of training labels indicating a health status, each training record includes an indication of a training patient, a timestamp, one or more training health measurements, and a training label indicating the health status of the training patient of the training record.

The server may obtain (e.g., receive or retrieve) a batch of patient data records from a database, which the server ingests as initial training records. The server then performs various operations for selecting or excluding data records to generate the training data records associated with training patients. The server generates the training data records according to various preconfigured selection parameters for pre-processing or conditioning the training data records in the initial training records.

In some implementations, the server selects the patient records of patients having preconfigured endpoints of time. The server assigns endpoints to the patient identifiers of patients to establish or configured sets of patients. The server may train the machine-learning architecture according to time limits or time bounds applied to the measurements of the patient records. The server randomly determines certain time endpoints or time intervals for patient records. For instance, during a training phase, the server randomly selects an endpoint for sampling measurements (e.g., BP values, BP trajectory) in the training records of the training patients. In some cases, the endpoint may be selected according to a health status indicators, such as an indicator that the training patient developed an instance HDP during the pregnancy. As an example, for the training patients who went on to develop HDP having an HDP instance in the status indicator, the server may randomly select endpoints to sample measurements between 20 weeks and 1 week prior the timestamp of the HDP diagnosis; for patients who did not go on to develop HDP, the server may randomly select endpoints to sample measurements between 20 weeks and 1 week prior to delivery at a conclusion timestamp of the pregnancy. In some implementations, the server uses 20 weeks as a lower bound for the endpoint so that training patients would have at least two in-clinic BP measures.

The server may implement any number of additional or alternative requirements for generating the training data records as described herein.

At operation 420, for each training record in the first training set having a first endpoint, the server extracts a set of training features. For each training patient, the server may parse or extract training measurements from the training records having timestamps that satisfy the first endpoint. The server may extract the features a feature vector or array of values. In some cases, the training features include the data values parsed from the training records, such as the measurement values of various types of measurements (e.g., BP measurements, systolic and diastolic values. In some cases, the server references the features or values of multiple training records of the training patient to generate derived features. Non-limiting examples of derived features may include summary statistics (e.g., maximum, minimum, mean, median), BP variability (e.g., average real variability; coefficient of variation), proportion of measures exceeding range, and slope (difference between earliest and latest BP divided by gestational age), among others.

At operation 430, for each training record in a second training set having a second endpoint, the server extracts a set of training features. For each training patient, the server may parse or extract training measurements from the training records having timestamps that satisfy the second endpoint.

At operation 440, training, by the computer, a machine-learning architecture of a risk prediction engine to generate a risk score using the set of training features and the training labels of the first training set of training records according to the first endpoint, the set of training features and the training labels of the second set of training records according to the second endpoint. The server may train or re-train the machine-learning model at predefined intervals or in response to receiving new training records for training patients. Likewise, at a predefined interval or in response to receiving updated patient records for the patient, server may generate new or updated features, derived features, and HDP risk score for the particular patient. The dynamic parameter variables in the machine-learning model may differentiate missing values between those from a later stage in pregnancy, which have yet to be collected, and uncollected values. In some implementations, training patients in the training data records include each variables corresponding to a prior timestamp of randomly selected gestational ages marked as “yet to be collected,” enabling the machine-learning model to learn associations relevant to predictions at each stage of pregnancy.

In some implementations, training labels associated with the training records indicate expected features, expected HDP risk score, or expected HDP health status. The server executes a loss function of a loss layer that references the training labels to determine a level of error between the predicted training features, predicted HDP risk score, or predicted HDP health status. The loss function may adjust or tune the parameters or variables of the machine-learning model of the machine-learning architecture to maximize certain outcomes or satisfy certain prediction accuracy training thresholds.

In some embodiments, the server implements logistic regression or Bayesian inference where the machine-learning architecture learns or tunes the model parameters by combining information from the training data with “prior” information. When learning the machine-learning model that incorporates at-home measurements, the machine-learning architecture considers the parameters as strong prior information. Likewise, when learning the machine-learning model that incorporates and parameters that incorporates the derived features of the at-home measurements, the machine-learning architecture considers the machine-learning model parameters as weakly informative prior distributions.

In some embodiments, rather than using a Bayesian approach, the server may update or retrain the parameters of the machine-learning models of the machine-learning architecture using stochastic gradient descent, with a slow-learning rate for parameters shared with the existing model, and a high learning rate for new, at-home measurements. In some embodiments, the machine-learning architecture includes a number of additive decision tree models. Such models learn a number of decision trees of restricted complexity, each of which is based on a subset of the training features. These tree models can naturally be augmented by maintaining the decision trees learned by the original model without at-home measurement data, and learning additional decision trees that include the at-home measurement features.

The server may determine the machine-learning model of the machine-learning architecture is trained in response to determining the level or error (or loss) satisfies a loss or training threshold. In some cases, after the model training is concluded, the server may evaluate or validate the trained machine-learning architecture using test patient records. In some cases, the server assess performance by race and ethnicity to ensure the model performs equitably and apply appropriate corrections (e.g. synthetic minority over-sampling technique).

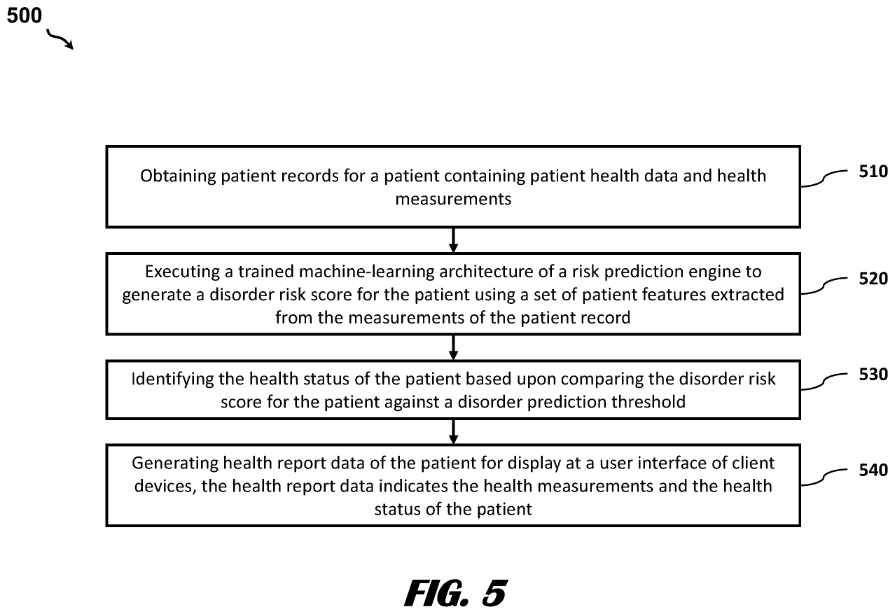

FIG. 5 shows operations of a computer-implemented method 500 for predicting potential instances of HDP according to patient data using a trained machine-learning model of a machine-learning architecture of a disorder prediction engine (e.g., disorder prediction engine 222), according to an embodiment. For ease of description and understanding, a server computer executes the various operations and features of the method 500, though embodiments are not so limited. Any number of computing devices may perform the operations and features of the method 500 for training the machine-learning architecture of the disorder prediction engine. Moreover, embodiments are not limited to the operations and features described in the method 500. Potential embodiments for training the machine-learning architecture of the disorder prediction engine may include any number of additional or alternative operations than those described in the method 500 and still fall within the scope of this disclosure.

At operation 510, at inference or deployment, the server obtains a plurality of patient records for a patient containing patient health data and one or more health measurements. For instance, the server receives a patient identifier and an operation instruction to generate an HDP risk score, HDP risk status indicator, or risk report data, among other potential requested operations. The server queries a database using the patient identifier of the current patient, and then selects or otherwise identifies the patient records having the patient identifier of the patient. The server uses these retrieved patient records as inputs to various operations and machine-learning layers of a machine-learning architecture for HDP prediction.

In some cases, the server generates HDP risk scores for patients having at least two measurements captured at different times. In such cases, the server executes validation operations to confirm that the server received patient records having a patient identifier of a patient, where the patient records include at least two measurements having at least two distinct timestamps. Other embodiments may implement additional or alternative data validation operations.

In some implementations, the server omits patient records having measurements that occurred within a threshold time from a prior HDP diagnosis. In such implementations, the server identifies a prior patient record in which the patient was diagnosed with HDP according to a health status indicator of the prior record. The server then compares a prior timestamp of the prior patient record against a current timestamp of a current or inbound patient record. The server removes, omits, or otherwise excludes the measurements of the current patient record in response to determining that the time-distance between the prior timestamp and the current timestamp fails the time threshold. In this way, the server excludes potential instances of “white coat” bias.

The server may implement any number of additional or alternative requirements for ingesting patient records and applying the machine-learning architecture on the patient data records, as described herein.

At operation 520, the server executes the machine-learning architecture of the risk prediction engine to generate the risk score for the patient using a set of patient features extracted from the one or more health measurements of the patient record.

The server may parse or extract training measurements from the training records having timestamps that satisfy the first endpoint. The server may extract the features a feature vector or array of values for the patient. In some cases, the features include the data values parsed from the patient records, such as the measurement values of various types of measurements (e.g., BP measurements, systolic and diastolic values. In some cases, the server references the features or values of multiple patient records of the patient to generate derived features. Non-limiting examples of derived features may include summary statistics (e.g., maximum, minimum, mean, median), BP variability (e.g., average real variability; coefficient of variation), proportion of measures exceeding range, and slope (difference between earliest and latest BP divided by gestational age), among others. As the database and/or the server obtains updated patient records, the server may update the extracted patient features and/or derived patient features of the patient to dynamically incorporate updated and ongoing measurements over the course of the pregnancy.

At operation 530, the server identifies the health status of the patient based upon comparing the HDP risk score for the patient against a disorder prediction threshold. The server predicts an instance of HDP for the patient's health status in response to determining that the HDP risk score of the patient satisfies the disorder prediction threshold. In some cases, the server executes a classifier layer of the machine-learning architecture as described herein for predicting the instance of the HDP and assigning HDP as the patient's health status. In some cases, the server may update the current patient record data to include an indication of the HDP instance as the health status.

At operation 540, the server generates a health report data of the patient for display at a user interface of one or more client devices, the health report data indicating the one or more health measurements and the health status of the patient. The health data report may have any file type or data structure that may be presented at a user interface of a client computer (e.g., patient computer, provider computer).

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, attributes, or memory contents. Information, arguments, attributes, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-Ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method of using machine-learning for predicting risks of hypertensive disorders of pregnancy (HDP), the method comprising:
generating, by a computer, a training dataset comprising a plurality of training records containing training health data for a plurality of patients and a corresponding plurality of training labels indicating a health status, each training record includes an indication of a training patient, a timestamp, one or more training health measurements, and a training label indicating the health status of the training patient of the training record;
for each training record in a first training set of one or more training patients having a first endpoint, extracting, by the computer, a set of training features from the one or more training measurements of the training record according to the first endpoint;
for each training record in a second training set of one or more training patients having a second endpoint, extracting, by the computer, the set of training features from the one or more training measurements of the training record according to the second endpoint;
training, by the computer, a machine-learning architecture of a risk prediction engine to generate a risk score using the set of training features and the training labels of the first training set of training records according to the first endpoint, the set of training features and the training labels of the second set of training records according to the second endpoint;
obtaining, by the computer, a plurality of patient records for a patient containing patient health data and one or more health measurements;
executing, by the computer, the machine-learning architecture of the risk prediction engine to generate the risk score for the patient using a set of patient features extracted from the one or more health measurements of the patient record;
identifying, by the computer, the health status of the patient based upon comparing the risk score for the patient against a disorder prediction threshold; and
generating, by the computer, health report data of the patient for display at a user interface of one or more client devices, the health report data indicating the one or more health measurements and the health status of the patient.

2. The method according to claim 1, further comprising selecting, by the computer, from the plurality of records of the training dataset the first training set of the training records having the first endpoint and the second training set of the training records having the second endpoint,
the timestamp of each training record in the first training set satisfying the first endpoint, and
the timestamp of each training record in the second training set satisfying the second endpoint.

3. The method according to claim 1, wherein each training record in the plurality of training records in the training dataset includes a source device indicator for a type of source device, including at least one of a clinician device or an edge device, and wherein the set of training features and the set of patient features includes the source device indicator for the type of source device.

4. The method according to claim 1, further comprising identifying, by the computer, in the training dataset a bias training record for the training patient, the health status of the bias training record indicating a disorder status for the training patient, and the timestamp of the bias training record fails to satisfy a status update threshold from the timestamp of a prior training record for the patient indicating an initial instance of the disorder status, wherein the computer omits each bias training record from the training dataset.

5. The method according to claim 1, wherein each training patient includes at least two training records having at least two training measurements.

6. The method according to claim 1, further comprising:
for a next training record for the training patient,
updating, by the computer, at least a portion of the set of training features for the training patient according to the one or more training measurements of the next training record; and
re-training, by the computer, the machine-learning architecture of the risk prediction engine to generate the risk score using the set of training features as updated and the training label of the next training record.

7. The method according to claim 1, further comprising:
for a next patient record for the patient,
extracting, by the computer, a set of updated features for the patient using the one or more measurements of the next patient record; and
generating, by the computer, an updated health report of the patient for display at the user interface of the one or more client devices.

8. The method according to claim 1, wherein the set of patient features for the patient and the set of training features for the training patient include at least one of: maximum blood pressure, minimum blood pressure, mean blood pressure, median blood pressure, blood pressure variability, blood pressure average real variability, blood pressure coefficient of variation, proportion of measures exceeding range, or a measurement slope.

9. The method according to claim 1, wherein the one or more measurements include at least one of: a systolic blood pressure, an interpregnancy interval, a prior diagnosis, a pre-pregnancy body mass index, a diastolic measurement, or a systolic measurement.

10. The method according to claim 1, further comprising in response to determining that the risk score for the patient satisfies the disorder prediction threshold, executing, by the computer, the machine-learning architecture of the risk prediction engine to identify one or more intervention actions corresponding to the set of one or more features extracted for the patient.

11. A system of using machine-learning for predicting risks of hypertensive disorders of pregnancy (HDP), the system comprising:
a computer comprising at least one processor and configured to:
generate a training dataset comprising a plurality of training records containing training health data for a plurality of patients and a corresponding plurality of training labels indicating a health status, each training record includes an indication of a training patient, a timestamp, one or more training health measurements, and a training label indicating the health status of the training patient of the training record;
for each training record in a first training set of one or more training patients having a first endpoint, extract a set of training features from the one or more training measurements of the training record according to the first endpoint;
for each training record in a second training set of one or more training patients having a second endpoint, extract the set of training features from the one or more training measurements of the training record according to the second endpoint;
train a machine-learning architecture of a risk prediction engine to generate a risk score using the set of training features and the training labels of the first training set of training records according to the first endpoint, the set of training features and the training labels of the second set of training records according to the second endpoint;
obtain a plurality of patient records for a patient containing patient health data, one or more health measurements, and a training label indicating the health status of the training patient for the training record;
execute the machine-learning architecture of the risk prediction engine to generate the risk score for the patient using a set of patient features extracted from the one or more health measurements of the training record;
identify the health status of the patient based upon comparing the risk score for the patient against a disorder prediction threshold; and
generate health report data of the patient for display at a user interface of one or more client devices, the health report data indicating the one or more health measurements and the health status of the patient.

12. The system according to claim 11, wherein the computer is further configured to:
select, from the plurality of records of the training dataset, the first training set of the training records having the first endpoint and the second training set of the training records having the second endpoint,
the timestamp of each training record in the first training set satisfying the first endpoint and the timestamp of each training record in the second training set satisfying the second endpoint.

13. The system according to claim 11, wherein each training record in the plurality of training records in the training dataset includes a source device indicator for a type of source device, including at least one of a clinician device or an edge device, and wherein the set of training features and the set of patient features includes the source device indicator for the type of source device.

14. The system according to claim 11, wherein the computer is further configured to:
identify in the training dataset a bias training record for the training patient, the health status of the bias training record indicating a disorder status for the training patient, and the timestamp of the bias training record fails to satisfy a status update threshold from the timestamp of a prior training record for the patient indicating an initial instance of the disorder status,
wherein the computer omits each bias training record from the training dataset.

15. The system according to claim 11, wherein each training patient includes at least two training records having at least two training measurements.

16. The system according to claim 11, wherein the computer is further configured to:
for a next training record for the training patient,
update at least a portion of the set of training features for the training patient according to the one or more training measurements of the next training record; and
re-train the machine-learning architecture of the risk prediction engine to generate the risk score using the set of training features as updated and the training label of the next training record.

17. The system according to claim 11, wherein the computer is further configured to:
for a next patient record for the patient,
extract a set of updated features for the patient using the one or more measurements of the next patient record; and
generate an updated health report of the patient for display at the user interface of the one or more client devices.

18. The system according to claim 11, wherein the set of patient features for the patient and the set of training features for the training patient include at least one of: maximum blood pressure, minimum blood pressure, mean blood pressure, median blood pressure, blood pressure variability, blood pressure average real variability, blood pressure coefficient of variation, proportion of measures exceeding range, or a measurement slope.

19. The system according to claim 11, wherein the one or more measurements include at least one of: a systolic blood pressure, an interpregnancy interval, a prior diagnosis, a pre-pregnancy body mass index, a diastolic measurement, or a systolic measurement.

20. The system according to claim 11, wherein the computer is further configured to in response to determining that the risk score for the patient satisfies the disorder prediction threshold, execute the machine-learning architecture of the risk prediction engine to identify one or more intervention actions corresponding to the set of one or more features extracted for the patient.
